(19) United States
(12) Patent Application Publication
(10) Pub. No.: US 2008/0319330 A1
(43) Pub. Date: Dec. 25, 2008

(54) TRANSMITTER AND RECEIVER FOR OBSERVING PERIODICAL EVENTS

(75) Inventors: Jouni JUNTUNEN, Helsinki (FI); Panu KEKALAINEN, Espoo (FI); Kauko PIENIMAKI, Helsinki (FI); Kalle Mannonen, Kirkkonummi (FI); Erik LINDMAN, Espoo (FI)

Correspondence Address: BIRCH STEWART KOLASCH & BIRCH, PO BOX 747, FALLS CHURCH, VA, 22040-0747, US

(73) Assignee: SUUNTO OY

(21) Appl. No.: 12/145,766

(22) Filed: Jun. 25, 2008

Related U.S. Application Data
(63) Continuation-in-part of application No. 11/169,712, filed on Jun. 30, 2005.
(60) Provisional application No. 60/585,225, filed on Jul. 2, 2004.

(51) Int. Cl. A61B 5/0404; H04B 7/00

(52) U.S. Cl. 600/509; 455/66.1

(57) ABSTRACT

A mobile transmitter for observing events and transmitting data on the observed events to a receiver and a device for receiving data messages from the mobile transmitter. The transmitter and receiver are designed so as to provide efficient and reliable transmission of data from the mobile transmitter to the receiver device.

### CROSS-REFERENCE TO RELATED APPLICATIONS

This application is a Continuation-In-Part of copending application Ser. No. 11/169,712 filed on Jun. 30, 2005, for which priority is claimed under 35 U.S.C. § 120, which is a non-provisional application claiming priority under 35 U.S.C § 119(e) on Provisional Application No. 60/585,225 filed on Jul. 2, 2004, the entire contents of which are hereby incorporated by reference.

### BACKGROUND OF THE INVENTION

The present invention relates to a mobile transmitter for observing events and transmitting data on the observed events to a receiver.

The invention also relates to a device for receiving data messages from a mobile transmitter.

Such mobile transmitters can be used, for example, in heart-rate monitors. The term heart-rate monitor refers also to a wristop computer or some other corresponding device, equipped with heart-rate monitor properties.

There are also various other fields of use for the mobile transmitters and receiver devices, such as speedometers, tachometers and any other devices adapted to monitor pulsed or periodically occurring events.

Heart-rate monitors and similar wristop computers typically include a transmitter belt attached to the human body by a flexible belt, which nowadays typically measures the pulse. This measuring device equipped with electrodes transmits a measuring message by radio to a wristwatch-like wristop computer, in which at least part of the received signal is processed and displayed on the display of the wristop computer. The wristop computers can be used to measure not only pulse, but also, for example, blood pressure, speed, acceleration, distance traveled, and direction data.

In the prior art mobile transmitter/receiver device solutions problems may arise in a situation in which information on one or some of the observed events is not transmitted to the receiver device. It may be very difficult for the receiver device to manage a situation, wherein detection information is missing. This lack of detection information may lead to confusion in the receiver device and to an error in the displayed information calculated on the basis of such information. On the other hand, there should not be extensive exchange of information between the mobile transmitter and the receiver device, e.g. due to limitations in size and power consumption of the devices.

### BRIEF SUMMARY OF THE INVENTION

Therefore, it is an object of the present invention to create a mobile transmitter and corresponding receiver device providing efficient and reliable transmission of data from the mobile transmitter to the receiver device.

According to an aspect of the invention, there is provided a mobile transmitter for observing events and transmitting data on the observed events to a receiver, the mobile transmitter comprising:
a detector for detecting occurrences of the observed events and responsive to the detection of each of said occurrences to generate a detection signal,
a timer for providing time references, and
a memory for recording time references,
   wherein the mobile transmitter is responsive to each detection signal to obtain a time reference from the timer and record the obtained time reference in the memory, and wherein the mobile transmitter is also operative to produce data messages containing a predetermined number of time references obtained from the memory and further to transmit the produced data messages to the receiver.

According to another aspect of the invention, there is provided a mobile transmitter for observing events and transmitting data on the observed events to a receiver, the mobile transmitter comprising:
a detector for detecting occurrences of the observed events and responsive to the detection of each of said occurrences to generate a detection signal,
a counter for counting detection signals,
a timer for providing time references, and
a memory for recording time references,
   wherein the mobile transmitter is adapted to count detection signals in the counter up to a predetermined count, and as response to reaching said predetermined count, adapted to obtain a time reference from the timer and record the obtained time reference in the memory and further to immediately start a new count of detection signals up to the predetermined count, and wherein the mobile transmitter is also operative to produce data messages containing a predetermined number of time references obtained from the memory and further to transmit the produced data messages to the receiver.

According to a further aspect of the invention, there is provided a device for receiving data messages from a mobile transmitter, wherein the received data messages contain a predetermined number of time references obtained from a timer of the mobile transmitter as responses to observed events detected by a detector of the mobile transmitter, the mobile device comprising:
a receiver for receiving the data messages, and
a data processor coupled to the receiver and operative to read and process the data messages in order to construct a sequence of consecutive time references relating to the observed events.

Therefore, the invention provides detection of the observed events and production of data messages containing predetermined number of time references indication the points in time wherein the observed events occurred. The mobile transmitter can proceed straightforwardly and does not need extensive processing power. Therefore, the mobile transmitter can be designed small and power-efficient. The receiver device receives time references and can base its calculations on accurate information.

According to embodiments, the time references include time values, so called time stamps. As the time stamps are transmitted to the receiver device, the receiver device can construct the sequence of time stamps relating to the original observed events. This sequence can then be used as a basis for different analyses, such as frequency analysis of the observed events. During such analyses, it is also possible to reconstruct missing time stamps by means of a mathematical pattern produced on the basis of the duly obtained and transmitted time stamps.

Furthermore, it is likely that the consecutive data messages contain overlapping information such that the same time references may occur in two or more of the data messages. This double-transmission of information provides reliability for the transmission, as loss of any single data message does not cause the receiver device being unaware of any of the time references. Thus, the receiver device receives a copy of every single time reference despite problems in transmitting some of the data messages. Such a double-transmission of information can be assured in embodiments wherein the number of time references in data messages and the time period between transmission of said messages are selected such that any one of the time references is included in at least two data messages even when the observing events occur at their maximum expected rate or frequency. However, enhanced reliability is attained already at lower retransmission rates than two. For example, the retransmission rate at the maximum expected rate or frequency of the observed events can be designed as 1.5 wherein enhanced reliability is attained also at the maximum rate. Even when the retransmission rate at the maximum expected rate or frequency of the observed events is about one, there is retransmission at the lower than the maximum rates of the observed events.

According to an embodiment, there is provided a heart-rate monitor comprising the above-referred mobile transmitter and device for receiving data messages from the mobile transmitter. In such an embodiment, the receiver device is included in a calculation and display unit of the heart-rate monitor and the mobile transmitter observes heart-beats and transmits data on the observed heart-beats to the calculation and display unit.

According to another embodiment, there is provided a tachometer for measuring a rate of revolution and comprising a mobile transmitter and a receiver device. According to a further embodiment, there is provided a speedometer for measuring speed of a wheel and comprising a mobile transmitter and a calculation unit as the receiver device.

According to an even further embodiment, there is provided a pace counter comprising a mobile transmitter and a calculation unit as the receiver device.

According to a further embodiment, there is provided a general event recorder for recording time data relating to observed events and comprising a mobile transmitter detecting said observed events and a receiver device recording the data on the observed events.

According to a further embodiment, there is provided a general event analyser comprising a mobile transmitter detecting observed events and a receiver device analysing the data on the observed events.

The observed event itself can be any event. Particularly suitable events are pulsed events that occur more or less sequentially whereby the events trigger pulses in the detector of the remote device. Examples of such pulsed events include heartbeats, revolutions and periodical sounds or light pulses. However, the observed event can be triggered by any measured variable reaching a threshold level. The observed events may occur occasionally or at approximately regular intervals.

In an embodiment, the mobile transmitter (the remote device) detects the observed events, such as heartbeats, and stores the time data relating to each event temporarily or semi-permanently in an internal memory. The time data is expressed in some mutually comparable form, for example, as a value given by an internal clock of the mobile transmitter, or some similar device expressing the passage of time. The time data can correspond to the local time of day, or be entirely independent of it. The time data should depict, with appropriate precision and mutually comparably, the moments in time at which the consecutive observed events occur. After this, the time data relating to the events are transmitted to the receiver device (terminal device), such as heart-rate monitor, wristop computer, or similar terminal device, along with identifier data. The measured variable, such as heart rate, pulse-interval data, speed or speed of rotation can now be calculated in the terminal device.

In an embodiment, the mobile transmitter has an identifier for identifying the transmitter in the receiver device. Such an identifier is included in every data message and the receiver device can disregard any messages received from neighbouring mobile transmitters. In such an embodiment, there is relatively little possibility of confusing the data transmitted between different terminal-device/remote-device pairs, (for example, a wristop-computer/transmitter-belt pair), and of internal confusion in a terminal-device/remote-device pair.

Compared to the prior art solutions, the above-mentions embodiment provides the advantages of both the easy transmission of sufficient data from a remote device (for example, a transmitter belt) to a terminal device (for example, a wristop computer) for it to be possible to calculate the measured variable, such as heart rate, and also of being able to determine if the data received is deficient.

An embodiment of a heart rate monitor permits the provision of very accurate heart rate data, pulse data or pulse-interval data to a wristop device or computer. Accurate pulse-interval data can be exploited, for example, when calculating variations of the pulse intervals, in a wristop device or computer, in exercise analysis. Pulse-interval data or variation in the pulse intervals can be used to obtain interesting information, for example, on the level of stress of the body.

In the following, the invention is examined with the aid of application examples according to the accompanying drawings.

### BRIEF DESCRIPTION OF THE DRAWINGS

FIG. 1 shows a block diagram of one device environment according to an embodiment.

FIG. 2 shows a block diagram of a mobile transmitter according to an embodiment.

FIG. 3 shows a block diagram of a receiver device according to an embodiment.

FIG. 4 shows one possible embodiment of a speedometer and heart rate monitor.

### DETAILED DESCRIPTION OF THE INVENTION

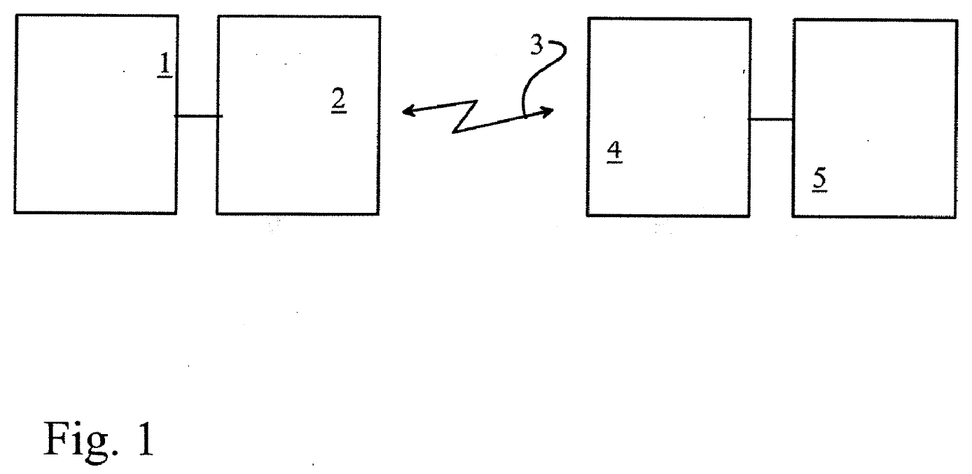

According to FIG. 1, the apparatus includes a measuring device 1, which is a pulse meter attached to the chest by a flexible belt. The pulse meter in question contains electrodes, with the aid of which the pulse of the person is measured. The measuring device 1 can naturally be some other measuring device. The measuring device is connected to a transmitter/coder 2, in which the measurement signal is edited into a transmittable form and given a code individuating the transmitter 2. The signal is sent from the transmitter 2 wirelessly over a transfer path 3 to a receiver 4, which also includes means for decoding the code. The transfer path 3 is typically the air space between the measuring device 2 located around the chest and a receiver 4 located on the wrist.

The receiver 4 is, in turn, connected to a data-processing unit 5, to which a display is typically also connected. The receiver 4 and the data-processing unit 5 are typically implemented in a wristop computer, which is reminiscent of a wristwatch. Such a wristop computer can include not only pulse-measuring properties or other measuring properties, but also normal clock functions, possibly positioning equipment, such as GPS circuits, and an altimeter, in which the sensor is typically a pressure sensor.

The wristop computer can also include, for example, a temperature measuring device. Also known are wristop computers with connections and data communications devices for connecting the wristop computer to a normal microcomputer, for example, through a USB bus.

The wristop computer or a similar device can also be designed such that it is adapted to be attached to sports equipment such as to a bicycle. The computer device can be attached, for example, to a handlebar of a bicycle.

In the following, methods according to the embodiments are described, for transmitting pulse data from a remote device to a terminal device. In such methods:
a message, which includes both pulse data and an identifier, is produced in the remote device, and
the message is transmitted from the remote device to the terminal device.

The pulse data may include, e.g. time data relating to heartbeats.

The identifier used in the embodiments can be in an analog form, or preferably in a digital form.

In one embodiment, the pulse data is coded into a digital form in the remote device, before transmission. In a second embodiment, the information contained in the message being transmitted to the terminal device is entirely coded into a digital form.

In the remote device, data from several observed events, such as heartbeats, can also be collected and the time data relating to the several events can be incorporated in a single message. The pulse data contained in each message can include, for example, a predefined number of time data, which relate to detected consecutive events. The number of time data contained in the message can be, for example, from 1 to 8, such as from 2 to 4, and preferably exactly 3.

The remote device produces the messages at regular intervals and includes in each of the messages the predefined number of the most current time data. The regular time interval can be selected such that it ensures sending each of the time data in at least two consecutive messages. In an embodiment, the number of time data contained in the messages is 3 and the time interval between the messages is 200 ms.

The message containing pulse data is produced in the remote device, without calculating the pulse-interval data. Thus the remote device does not necessarily need a functionality of this kind at all.

The identifier in a digital form contained in the message individuates the remote device, allowing the terminal device to identify the remote device with the aid of the digital-form identifier.

Further, according to an embodiment, the time data includes a value obtained from a time reference, which is selected on the basis of the moment of detecting the observed event. This typically takes place by the time value being retrieved from the time reference immediately after the detection of the event. The time value is thus received after an operating delay of the device from the detection of the event. As the device's operating delay remains essentially constant, the time values obtained will be mutually comparable, as the same operating delay relates to each time value. The time reference used is typically an internal clock device in the remote device.

Thus the values obtained from the time reference can be relative, as they express the moment of the event relative to the moment of the preceding or following events. Alternatively, the values obtained from the time references can be essentially absolute, by expressing with substantial accuracy the moment of the event in local time.

It should be further stated that, in the most usual embodiment, the messages are sent wirelessly using a radio frequency.

The aforementioned operations can thus be performed in a remote device, for example, in the transmitter part of a pulse meter. In the terminal device, for example, in the calculation and display unit of the pulse meter, the operations depicted in the following can, for their part, be performed.

According to the heart rate monitor embodiments, the terminal device receives messages containing pulse data and an identifier, sent by the remote device and defines the current heart ratevalue on the basis of the pulse data contained in the received messages. In such embodiments, the pulse data comprises time data relating to the heartbeats.

The identifier used in the embodiments can be in an analog form, or preferably in a digital form.

In one embodiment, the pulse data received is coded into a digital form. In a second embodiment, the information contained in the received message is entirely coded into a digital form.

The received message can include time data relating to one or more events. In some embodiments, the pulse data contained in each message comprises a predefined number of time data, which relate to consecutive events detected in the remote device. The number of time data contained in the message can be, for example, from 1 to 8, such as from 2 to 4, and preferably exactly 3.

In an embodiment providing information on the intervals between the observed events, the interval information need not be calculated in the remote device. Instead, the interval data can be calculated in the terminal device such as a wristop computer on the basis of the received time data, if information is required on the time that has elapsed between the observed events.

To prevent confusion in the terminal device between different pairs of terminal-devices/remote-devices, it is advantageous to examine the digital-form identifier contained in each message and, if the identifier differs from the identifier set in the terminal device, to reject a message containing a differing identifier.

Thus, each time datum can include a relative time value, so that the time values express the moment of the observed event, relative to the moment of the preceding or following observed events, or an essentially absolute time value, so that the time values express, with substantial accuracy, the moment of the observed event in local time.

The message is typically received wirelessly using a radio frequency.

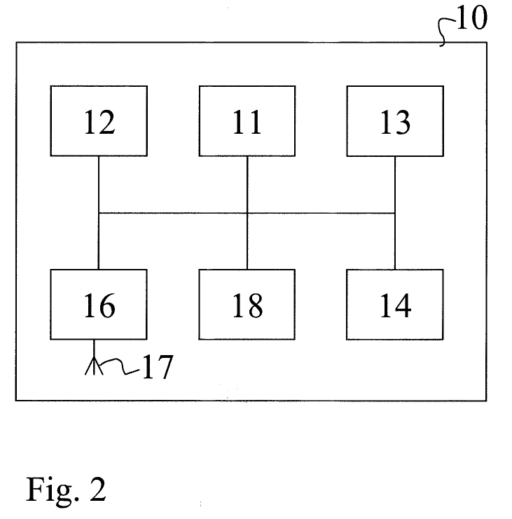

FIG. 2 shows a mobile transmitter 10 for observing events and transmitting data on the observed events to a receiver. The mobile transmitter 10 of FIG. 2 further comprises a controller 11, which can be a microcontroller, ASIC or a general purpose microprocessor, for instance. The controller 11 controls the operations of the mobile transmitter 10.

The mobile transmitter 10 of FIG. 2 further comprises a detector 12 for detecting occurrences of the observed events. Such a detector 12 is coupled to the controller 11 and can be, for example, a photoelectric sensor, an electromagnetic sensor, a pressure sensor, an acoustic sensor, an EMG detector or any other sensor capable of detecting the observed events and providing a detection signal for the controller 12 as response to the detection of the events.

The mobile transmitter 10 of FIG. 2 further comprises a timer 13 which is also coupled to the controller 11 or formed as part of the same microchip. Thus, the internal clock of the controller 11 may act as the timer 13 as well. The timer 13 provides time references when requested by the controller 11.

The mobile transmitter 10 of FIG. 2 further comprises a memory 14 for recording the time references. The data storage requirement of the memory 14 is very limited and therefore the memory 14 may well be formed by an internal memory of the controller 11 chip. Of course, it is possible to provide the mobile transmitter 10 with a separate memory chip but this is often not necessary in case the controller 11 itself already contains a memory. The memory 14 can be operated on a last-in-first-out basis in order to temporarily store the predefined number of most current time data. The memory can also be operated on a first-in-first-out basis wherein the memory 14 has its number of memory locations designed to equal the predefined number of time data to be included in each of the transmitted messages. In addition to the memory locations reserved for the memory 14, the mobile transmitter 10 contains further memory locations for storing the appropriate control logics and parameters, such as the predefined number and the time interval.

According to a further embodiment, the memory 14 for recording the time references has a larger capacity for semi-permanent storage of time data. Then the memory 14 is capable of storing time data relating to longer periods of time. In such an embodiment the time data can be read from the memory afterwards instead of, or in addition to, the wireless transmission to the receiver device.

The mobile transmitter 10 of FIG. 2 further comprises a radio transmitter 16 and an antenna 17 for transmitting the messages formed by the mobile transmitter 10. The mobile transmitter 10 also comprises a battery 18 or other source of operation energy.

The controller 11 is adapted to control the mobile transmitter 10 such that it is responsive to each detection signal to obtain a time reference from the timer 13 and record the obtained time reference in the memory 14. The controller 11 is further adapted to produce data messages containing a predetermined number of time references obtained from the memory 14 and further to submit them to the radio transmitter 16 for transmission to a receiver.

In an embodiment, the detector 12 comprises a circuit for detecting magnetic pulses.

In an embodiment, the detector 12 comprises a circuit for detecting electrical pulses.

In an embodiment, the detector 12 is configured to detect a heartbeat of a human body.

In an embodiment, the detector 12 comprises an acceleration sensor.

In an embodiment, the mobile transmitter 10 is operative to produce and transmit the data messages at predetermined time intervals. In some embodiments, the controller 11 may alter the length of the predetermined time interval, for example, responsive to the frequency of detected events. In another embodiment enabling a very compact construction of the mobile transmitter 10, the predetermined time intervals have a common constant value that is permanently set in the mobile transmitter 10.

In an embodiment, the common constant value of the predetermined time intervals is selected from between 50 to 1000 ms and the predetermined number of time references in the data messages is from 1 to 10.

In another embodiment, the common constant value of the predetermined time intervals is selected from between 150 to 400 ms and the predetermined number of time references in the data messages is from 2 to 4.

In a particular embodiment, the common constant value of the predetermined time intervals is 200 ms and the predetermined number of time references in the data messages is 3.

In general, the number of time references in the data messages N and the time interval T_{message} can be selected such that

$$\frac{N}{{T}_{message}} \geq \frac{C}{{T}_{event}}$$

wherein Tₑᵥₑₙₜ is the minimum expected period between the observed events and C is the double-transmission factor that indicates how many copies of the time references should at least be sent. The C can be designed, for example, such that it is at least 2 wherein a loss of any single message does not lead to a loss of information. In case the operating environment is noisy or for other reasons even more reliability is desired, the factor C can be selected to be, for example, at least 3 or at least 4 or 5. However, in order to optimize the power consumption of the mobile transmitter 10, it is beneficial to avoid designing the factor C too high, at least in application wherein the battery life is critical. Values of factor C between 1.5 and 3 may well be sufficient in application wherein some instantaneous measuring errors can be accepted and excessive power consumption is avoided.

In an embodiment, the mobile transmitter 10 is adapted to observe a system exhibiting periodically occurring events having a presumed minimum interval Tₑᵥₑₙₜ, and wherein the predetermined interval T_{message} between the successive data messages and the predetermined number N of time references in each of the data messages have been selected in the mobile transmitter 10 such that each of the obtained time references will be included in at least two of the consecutive data messages when detecting events occurring at the presumed minimum intervals.

In case it is expected that the frequency of the observed events is high and information on the every single moment of occurrence is not critical, the mobile transmitter 10 can be constructed such that it provides a time reference for a particular number of events instead of every single event. Then, the receiver device can take this into account in its calculations by using a corresponding multiplier, when necessary. For example, the mobile transmitter 10 may be adapted to provide time references for every second or tenth detected event and the receiver device can calculate the rate of the observed events by multiplying the number of time references by two or ten, respectively. The multiplier can, of course, be freely selected according to the application when designing the device or even by the device setting in case the device provides such functionality. Examples of high frequency applications suitable for such embodiments may be, for example, measuring a speed of a motor vehicle or a tachometer in an engine.

The above-referred high frequency transmitter can be formed by providing the mobile transmitter with a counter for counting detection signals and configuring the mobile transmitter to count detection signals in the counter up to a predetermined count, and always when reaching said predetermined count, to obtain a time reference from the timer and record the obtained time reference in the memory. Furthermore, the mobile transmitter is configured to immediately after each time reference to start a new count of detection signals up to the predetermined count.

In an embodiment, the mobile transmitter 10 is operative to produce the data messages such that each of the data messages contains the predetermined number of the latest time references in the memory 14.

In an embodiment, the mobile transmitter 10 is operative to produce the data messages periodically such that a predetermined interval of time lapses between each of the successive data messages.

In an embodiment, the mobile transmitter 10 has an identifier and the controller 11 is adapted to include said identifier in each of the produced data messages.

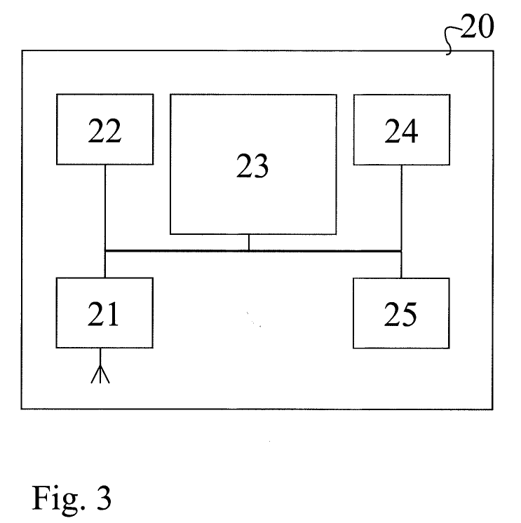

FIG. 3 shows a device 20 for receiving data messages from the mobile transmitter 10 of FIG. 2. Therefore, the received data messages contain a predetermined number of time references obtained from the timer 13 of the mobile transmitter 10 as responses to observed events detected by the detector 12. The receiver device 20 comprises a receiver 21 for receiving the data messages. The receiver 21 is a suitable radio receiver. The receiver device 20 further comprises a data processor 22 coupled to the receiver 21 and operative to read and process the data messages in order to construct a sequence of consecutive time references relating to the observed events. Furthermore, the receiver device 20 comprises the necessary auxiliary devices such as a display 23, memory 24 and power source 25.

In an embodiment, the data processor 22 is adapted to operate the receiver device 20 so that it disregards each further occurrence of a same time reference when constructing the sequence of consecutive time references.

In an embodiment, the data processor 22 is adapted to calculate, on the basis of said sequence of consecutive time references, a pulse rate of the observed events.

In an embodiment, the data processor 22 is adapted to calculate, on the basis of said sequence of consecutive time references, time intervals between the observed events. In an embodiment, the data processor 22 is adapted to calculate, on the basis of said sequence of consecutive time references, variations of the time intervals between the observed events.

In an embodiment wherein each of the received data messages contain an identifier identifying the transmitter of said message, the data processor 22 can disregard data messages containing a wrong identifier.

In a further embodiment each of the received data messages contain an identifier identifying the transmitter of said message and the receiver devices is adapted to utilize information received from a plurality of mobile transmitters. Then, the data processor 22 can distinguish the sources of time references by means of the identifiers and thereby use correct time information in calculating the measured variables.

Embodiments of the mobile transmitter 10 of FIG. 2 and the receiver device 20 of FIG. 3 can be utilized is various applications.

According to an embodiment, the mobile transmitter 10 and the receiver device 20 are used to construct a heart-rate monitor. The heart-rate monitor comprised a calculation and display unit and a mobile transmitter for observing heart-beats and transmitting data on the observed heart-beats to the calculation and display unit. The calculation and display unit is an embodiment of the receiver device 20 and the mobile transmitter is an embodiment of the mobile transmitter 10 of FIG. 2.

According to the embodiment of the heart-rate monitor, a detector detects the heartbeats and generates detection signals responsive to the detection of the heartbeats. A timer provides time references and they are temporarily stored in a memory. The mobile transmitter produces data messages containing an identifier and a predetermined number of time references obtained from the memory and transmits the messages to the calculation and display unit. The calculation and display unit receives the data messages constructs a sequence of consecutive time references relating to the heartbeats. Furthermore, the calculation and display unit calculates and displays at least one of a pulse rate, intervals of the heartbeats and variations of the intervals of heartbeats.

According to a further embodiment, the mobile transmitter of the heart-rate monitor is attachable to the human body by means of a flexible belt, and the calculation and display unit comprises a wristband and is attachable to the human body by the wristband. Furthermore, the mobile transmitter is adapted to transmit the data messages to the calculation and display unit wirelessly as a radio frequency transmission.

According to a further embodiment of the heart-rate monitor, the mobile transmitter is operative to produce the data messages periodically such that a predetermined interval of time lapses between each of the successive data messages and both the predetermined interval between the successive data messages and the predetermined number of time references in each of the data messages have been selected such that each of the obtained time references will be included in at least two of the consecutive data messages when detecting heart-beats at a presumed maximum heart rate of a human being.

In the heart-rate monitor embodiments, the predetermined number of time references in the data messages is usually between 2 and 8 and data messages are sent at constant interval of 50 to 1000 ms.

In a particular embodiment, the number of time references in the data messages is 3 and the time interval between the messages is about 200 ms, and more generally in the range of 150 to 400 ms.

According to a further embodiment, the mobile transmitter 10 and the receiver device 20 are used to construct a tachometer for measuring a rate of revolution of a wheel or any other rotation object. The wheel can be a wheel of a bicycle, for instance. In such an embodiment, the mobile transmitter detects the revolutions of the object and provides time references relating to such revolutions. The mobile transmitter submits data messages containing an identifier and a predetermined number of time references to a calculation unit, which calculates the rate of revolution on the basis of the received time references.

In an embodiment of the tachometer, the detector is adapted to detect magnetic pulses caused by a magnet periodically passing the detector. The magnet is attached to the rotating object such as a wheel.

In a further embodiment of the tachometer, the detector comprises an optical sensor for detecting revolutions. Such detection can be based on a reflection by a reflector, detection of a laser beam or light passing a slot, contrast or optical pattern in the object or any other optical sign indicating a revolution.

According to a further embodiment, the mobile transmitter 10 and the receiver device 20 are used to construct a speedometer for measuring speed of a wheel. This embodiment can be designed very much alike the tachometer embodiment but is adapted to calculate the speed of the wheel on the basis of the time references. The speed of the wheel can be calculated by multiplying the rate of revolution by the perimeter of the wheel.

According to a further embodiment, the mobile transmitter 10 and the receiver device 20 are used to construct a pace counter for counting steps of a walking or running person. In addition to bare pace count, the embodiment is capable of providing a time reference relating to each step and therefore the calculation unit can calculate various parameters relating to the exercise.

According to an even further embodiment, the mobile transmitter 10 and the receiver device 20 are used to construct a counter for counting strokes in sports or fitness equipment. Also this embodiment can provide accurate time information relating to each stroke and thus basis for thorough analysis of the exercise.

In an even further embodiment, the receiver device 20 is adapted to receive information from a plurality of mobile transmitters 10 measuring different parameters. Therefore, it is possible to receive information on two or several of heartbeat, rate of revolution, speed, pace count, stroke count and other variables and calculate and display the measured variables in a single receiver device and in a single display. Furthermore, the receiver device 20 can correlate the measured variables and thus make or provide basis for more sophisticated analysis of an exercise.

The receiver device 20 can also receive information from a plurality of mobile transmitters 10 measuring different objects such as a group of people. In such an embodiment, the receiver device 20 can distinguish the transmitters with the aid of identifiers and calculate own variables for each person of the group.

Such receiver devices 20 providing more sophisticated functionalities can be wristop computers or laptop computers, for instance.

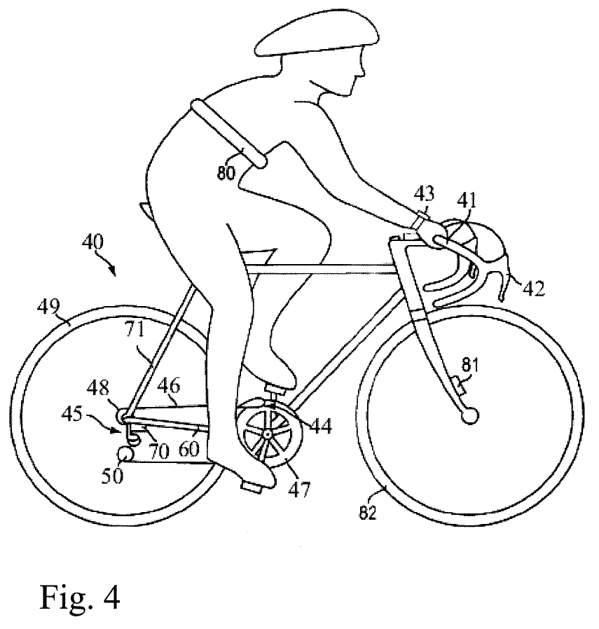

FIG. 4 depicts one possible application for the mobile transmitter and the receiver device. The embodiment of FIG. 4 is a system for monitoring running parameters of a bicycle. FIG. 4 shows a bicycle 40 that comprises a handlebar 41 on which are disposed brake handles 42 and control for front derailleurs 44 and rear derailleurs 45. FIG. 4 shows also a chain 46, gear wheels 47, sprockets 48, a rear wheel 49 and return means 50, a frame 60, a receiver/transmitter 70, a heart rate meter 80, a detector 81 and a front wheel 82. The detector 81 is adapted to detect revolutions of the front wheel 82. The system can also be provided with a detector for detecting pedaling cadence.

FIG. 4 shows also a wristop computer 43 which can act as a receiver device for data messages transmitted by the heart rate meter 80 and the detector 81 acting as mobile transmitters. Therefore, the system of FIG. 4 can be used to measure the heart rate of the cycler, together with the pedaling cadence and speed, for instance. These variables can be further analyzed in order to provide further information on the exercise. Furthermore, the measured variables may be correlated with further information measured or obtained by the wristop computer 43, such as altitude, temperature and/or location.

The system of FIG. 4 can also be provided with a small computer mounted on the handlebar 41. This computer can act as the receiver of the transmitted time data instead of, or in addition to, the wristop computer 43. The small computer mounted on the handlebar 41 may also display the running parameters of the bicycle, possibly together the other information such as heart rate, to the cycler during cycling. Furthermore, the computer may be programmed to give guidance to the cycler as to selection of the gears and suitable pedaling cadence, for instance.

Systems for monitoring running parameters of a bicycle and controlling a bicycle are described in detail in U.S. Pat. No. 6,779,401, Bruno Montagnion, and U.S. Pat. No. 6,204,775, Michel Kubacsi, both of which patents are incorporated herein by reference. The above-described embodiments of the mobile transmitter and receiver device can be applied also in the embodiments described in these two incorporated patents for transmitting information between the different parts of the systems.

The invention being thus described, it will be obvious that the same may be varied in many ways. Such variations are not to be regarded as a departure from the spirit and scope of the invention, and all such modifications are intended to be included within the scope of the following claims.

1. A mobile transmitter for observing events and transmitting data on the observed events to a receiver, the mobile transmitter comprising:
a detector for detecting occurrences of the observed events and responsive to the detection of each of said occurrences to generate a detection signal,
a timer for providing time references, and
a memory for recording time references,
wherein the mobile transmitter is responsive to each detection signal to obtain a time reference from the timer and record the obtained time reference in the memory, and wherein the mobile transmitter is also operative to produce data messages containing a predetermined number of time references obtained from the memory and further to transmit the produced data messages to the receiver.

2. A mobile transmitter for observing events and transmitting data on the observed events to a receiver, the mobile transmitter comprising:
a detector for detecting occurrences of the observed events and responsive to the detection of each of said occurrences to generate a detection signal,
a counter for counting detection signals,
a timer for providing time references, and
a memory for recording time references,
wherein the mobile transmitter is adapted to count detection signals in the counter up to a predetermined count, and as response to reaching said predetermined count, adapted to obtain a time reference from the timer and record the obtained time reference in the memory and further to immediately start a new count of detection signals up to the predetermined count, and wherein the mobile transmitter is also operative to produce data messages containing a predetermined number of time references obtained from the memory and further to transmit the produced data messages to the receiver.

3. The mobile transmitter of claim 1 or 2, wherein the mobile transmitter is operative to produce and transmit the data messages at predetermined time intervals.

4. The mobile transmitter of claim 3, wherein each of the predetermined time intervals have a common constant value.

5. The mobile transmitter of claim 4, wherein the common constant value of the predetermined time intervals is in the range of 50 to 1000 ms and the predetermined number of time references in the data messages is from 1 to 10.

6. The mobile transmitter of claim 4, wherein the common constant value of the predetermined time intervals is in the range of 150 to 400 ms.

7. The mobile transmitter of claim 6, wherein the predetermined number of time references in the data messages is from 2 to 4.

8. The mobile transmitter of claim 1 or 2, wherein the mobile transmitter is operative to produce the data messages such that each of the data messages contains the predetermined number of the latest time references in the memory.

9. The mobile transmitter of claim 8, wherein the mobile transmitter is operative to produce the data messages periodically such that a predetermined interval of time lapses between each of the successive data messages.

10. The mobile transmitter of claim 9, wherein the mobile transmitter is adapted to observe a system exhibiting periodically occurring events having a presumed minimum interval, and wherein the predetermined interval between the successive data messages and the predetermined number of time references in each of the data messages have been selected in the mobile transmitter such that each of the obtained time references will be included in at least two of the consecutive data messages when detecting events occurring at the presumed minimum intervals.

11. A receiver device for receiving data messages from a mobile transmitter, wherein the received data messages contain a predetermined number of time references obtained from a timer of the mobile transmitter as responses to observed events detected by a detector of the mobile transmitter, the receiver device:
a receiver for receiving the data messages, and
a data processor coupled to the receiver and operative to read and process the data messages in order to construct a sequence of consecutive time references relating to the observed events.

12. The receiver device of claim 11, wherein the mobile device is adapted to disregard each further occurrence of a same time reference when constructing the sequence of consecutive time references.

13. The receiver device of claim 11, wherein the data processor is adapted calculate, on the basis of said sequence of consecutive time references, at least one of a pulse rate of the observed events, intervals of the observed events and variations of the intervals of the observed events.

14. The receiver device of claim 11, wherein each of the received data messages contain an identifier identifying the transmitter of said message and the mobile device is adapted to disregard data messages containing a wrong identifier.

15. A heart-rate monitor, comprising a calculation and display unit and a mobile transmitter for observing heart-beats and transmitting data on the observed heart-beats to the calculation and display unit,
wherein the mobile transmitter comprises:
a detector for detecting the heart-beats, the detector being responsive to the detection of each of heart-beats to generate a detection signal,
a timer for providing time references, and
a memory for recording time references,
wherein the mobile transmitter is responsive to each detection signal to obtain a time reference from the timer and record the obtained time reference in the memory, and wherein the mobile transmitter is also operative to produce data messages containing an identifier and a predetermined number of time references obtained from the memory and further to transmit the produced data messages to the calculation and display unit, and
wherein the calculation and display unit comprises:
a receiver for receiving the data messages,
a data processor coupled to the receiver and operative to construct a sequence of consecutive time references relating to the observed events on the basis of said data messages and to calculate, on the basis of said sequence of consecutive time references, at least one of a pulse rate, intervals of the heart-beats and variations of the intervals of heart-beats, and
a display for displaying at least one of the calculated variables.

16. The heart-rate monitor of claim 15, wherein the mobile transmitter is attachable to the human body by means of a flexible belt, and the calculation and display unit comprises a wristband and is attachable to the human body by the wristband, and wherein the mobile transmitter is adapted to transmit the data messages to the calculation and display unit wirelessly as a radio frequency transmission.

17. The heart-rate monitor of claim 15, wherein the mobile transmitter is operative to produce the data messages periodically such that a predetermined interval of time lapses between each of the successive data messages.

18. The heart-rate monitor of claim 17, wherein both the predetermined interval between the successive data messages and the predetermined number of time references in each of the data messages have been selected such that each of the obtained time references will be included in at least two of the consecutive data messages when detecting heart-beats at a presumed maximum heart rate of a human being.

19. The heart-rate monitor of claim 15, wherein the predetermined number of time references in the data messages is from 2 to 8.

20. The heart-rate monitor of claim 15, wherein the predetermined number of time references in the data messages is 3.

21. The heart-rate monitor of claim 17, wherein said predetermined interval of time is a constant value selected from the range of 50 to 1000 ms

22. The heart-rate monitor of claim 17, wherein said predetermined interval of time is a constant value selected from the range of 150 to 400 ms.

23. A tachometer for measuring a rate of revolution, comprising a calculation unit and a mobile transmitter for transmitting data to the calculation unit,
wherein the mobile transmitter comprises:
a detector for detecting a revolution, the detector being responsive to the detection of each revolution to generate a detection signal,
a timer for providing time references, and
a memory for recording time references,
wherein the mobile transmitter is responsive to each detection signal to obtain a time reference from the timer and record the obtained time reference in the memory, and wherein the mobile transmitter is also operative to produce data messages containing an identifier and a predetermined number of time references obtained from the memory and further to transmit the produced data messages to the calculation unit, and
wherein the calculation unit comprises:
a receiver for receiving the data messages, and
a data processor coupled to the receiver and operative to construct a sequence of consecutive time references relating to the revolutions on the basis of said data messages and to calculate the rate of revolution on the basis of said sequence of consecutive time references.

24. A tachometer for measuring a rate of revolution, comprising a calculation unit and a mobile transmitter for transmitting data to the calculation unit,
wherein the mobile transmitter comprises:
a detector for detecting a revolution, the detector being responsive to the detection of each revolution to generate a detection signal,
a counter for counting detection signals,
a timer for providing time references, and
a memory for recording time references,
wherein the mobile transmitter is adapted to count detection signals in the counter up to a predetermined count, and as response to reaching said predetermined count, adapted to obtain a time reference from the timer and record the obtained time reference in the memory and further to immediately start a new count of detection signals up to the predetermined count, and wherein the mobile transmitter is also operative to produce data messages containing an identifier and a predetermined number of time references obtained from the memory and further to transmit the produced data messages to the calculation unit, and
wherein the calculation unit comprises:
a receiver for receiving the data messages, and
a data processor coupled to the receiver and operative to construct a sequence of consecutive time references relating to the revolutions on the basis of said data messages and to calculate the rate of revolution on the basis of said sequence of consecutive time references.

25. A speedometer for measuring speed of a wheel, the speedometer comprising a calculation unit and a mobile transmitter for transmitting data to the calculation unit,
wherein the mobile transmitter comprises:
a detector for detecting revolutions of the wheel, the detector being responsive to the detection of each revolution to generate a detection signal,
a timer for providing time references, and
a memory for recording time references,
wherein the mobile transmitter is responsive to each detection signal to obtain a time reference from the timer and record the obtained time reference in the memory, and wherein the mobile transmitter is also operative to produce data messages containing an identifier and a predetermined number of time references obtained from the memory and further to transmit the produced data messages to the calculation unit, and
wherein the calculation unit comprises:
a receiver for receiving the data messages, and
a data processor coupled to the receiver and operative to construct a sequence of consecutive time references relating to the revolutions on the basis of said data messages and to calculate the speed of the wheel on the basis of said sequence of consecutive time references.

26. A speedometer for measuring speed of a wheel, the speedometer comprising a calculation unit and a mobile transmitter for transmitting data to the calculation unit,
wherein the mobile transmitter comprises:
a detector for detecting revolutions of the wheel, the detector being responsive to the detection of each revolution to generate a detection signal,
a counter for counting detection signals,
a timer for providing time references, and
a memory for recording time references,
wherein the mobile transmitter is adapted to count detection signals in the counter up to a predetermined count, and as response to reaching said predetermined count, adapted to obtain a time reference from the timer and record the obtained time reference in the memory and further to immediately start a new count of detection signals up to the predetermined count, and wherein the mobile transmitter is also operative to produce data messages containing an identifier and a predetermined number of time references obtained from the memory and further to transmit the produced data messages to the calculation unit, and
wherein the calculation unit comprises:
a receiver for receiving the data messages, and
a data processor coupled to the receiver and operative to construct a sequence of consecutive time references relating to the revolutions on the basis of said data messages and to calculate the speed of the wheel on the basis of said sequence of consecutive time references.

27. A pace counter, comprising a calculation unit and a mobile transmitter for transmitting data to the calculation unit,
wherein the mobile transmitter comprises:
a detector for detecting a step, the detector being responsive to the detection of each step to generate a detection signal,
a timer for providing time references, and
a memory for recording time references,
wherein the mobile transmitter is responsive to each detection signal to obtain a time reference from the timer and record the obtained time reference in the memory, and wherein the mobile transmitter is also operative to produce data messages containing an identifier and a predetermined number of time references obtained from the memory and further to transmit the produced data messages to the calculation unit, and
wherein the calculation unit comprises:
a receiver for receiving the data messages, and
a data processor coupled to the receiver and operative to construct a sequence of consecutive time references relating to the steps on the basis of said data messages and to calculate the pace count on the basis of said sequence of consecutive time references.
